(19) United States
(12) Patent Application Publication
(10) Pub. No.: US 2010/0006104 A1
(43) Pub. Date: Jan. 14, 2010

(54) Tracheostomy tie system and method

(76) Inventor: James L. Leckie, Covina, CA (US)

Correspondence Address: KOPPEL, PATRICK, HEYBL & DAWSON, 2815 Townsgate Road, SUITE 215, Westlake Village, CA, 91361-5827, US

(21) Appl. No.: 12/381,081

(22) Filed: Mar. 5, 2009

Related U.S. Application Data
(63) Continuation-in-part of application No. 12/170,255, filed on Jul. 9, 2008.

(51) Int. Cl. A61M 16/00

(52) U.S. Cl. 128/207.17; 112/475.08

(57) ABSTRACT

A tracheostomy tie includes a padded collar with a surface engagement side, a strap coupled to the padded collar on a side opposite from the surface engagement side and extending from opposite ends of the padded collar. The strap includes a loop material outer surface and tapered distal ends. Connectors tapered at both distal ends are connected to respective tapered distal ends of the strap, each of the tapered connectors having a hook material outer surface. The tapered connectors are operable to pass through respective tracheostomy tube connectors and loop back to engage the loop material so the padded collar may securely fastens and positions a tracheostomy tube.

This application is a continuation in part and claims the benefit of U.S. patent application Ser. No. 12/170,255 to James L. Leckie, which was filed on 9 Jul. 2008.

### BACKGROUND OF THE INVENTION

1. Field of the Invention

This invention relates to medical devices, and more particularly to ties to secure a tracheostomy tube to a patient's neck.

2. Description of the Related Art

Patients who have undergone a tracheostomy require a tracheal tube to be securely and comfortably attached to their neck over long periods of time. A tracheal tube assembly is inserted into a hole created in the patient's trachea and often secured to the patient's neck using a tracheostomy tie. Typical tracheostomy ties consist of a variety of materials, such as foam padding, elastic cloth, and wide and narrow materials to maintain a secure fit about the patient's neck while attempting to reduce patient discomfort.

A tracheostomy tie will typically pass around the patient's neck to secure and support the tracheal tube assembly, with each end of the tracheostomy tie fed through eyelets incorporated into either side of the tracheal tube assembly. The ends of the tracheostomy tie often incorporate the use of VELCRO™ material to fold back on itself to secure the tie to the tracheostomy tube assembly.

However, feeding the ends of a tracheostomy tie through the eyelets on either side of the tracheal tube can prove to be difficult, particularly in common medical scenarios where the tracheal tube must be secured by the tie as quickly as possible. Moreover, because of the need for constant supervision and care for patients using tracheostomy tube assemblies, there is often only one caregiver at a time, making rapid change out of the tracheostomy tie difficult with only two hands to both hold the tracheostomy tube assembly and replace the tie. Also, the VELCRO™ or similar material used to secure the tie in place often comes undone via movement of the patient and/or lack of appropriate surface area for securing the ends of the tie back onto the tie assembly.

Additionally, long term use of the tracheostomy tie around the patient's neck produces a breakdown of the skin underneath the tracheostomy tie. Moisture produced at the skin/tie interface accelerates breakdown of the skin, in part because of stress-strain deformation of the tie about the patient's neck over time; this can cause localized chafing of the patient's skin. Gauze material is often used between the tracheostomy tube and the patient's skin to reduce this effect by absorbing moisture and increasing the surface area supporting the weight of the tracheostomy tie and tracheostomy tube assembly.

Therefore, a need exists to reduce the breakdown of patient skin underneath the tracheostomy tie and to reduce the difficulty associated with securing and replacing the tracheostomy tie.

### SUMMARY OF THE INVENTION

The present invention seeks to provide a tracheostomy tie and method that allows for a tracheostomy tube assembly to be efficiently, comfortably, and securely attached to a patient. One embodiment according to the present invention comprises a tracheostomy tie apparatus with a padded collar having a surface engagement side, and a strap coupled to the padded collar on a side opposite from the surface engagement side and extending from opposite ends of the padded collar. The strap comprises a loop material outer surface and tapered distal ends and one or more connectors tapered at both distal ends, with the connectors coupled to respective tapered distal ends of the strap. Each of the tapered connectors has a hook material outer surface. One or more connectors are operable to pass through respective tracheostomy tube connectors and loop back to engage the loop material so the padded collar securely fastens and positions a tracheostomy tube.

Pursuant to another embodiment, a tracheostomy tie apparatus is provided comprising a padded collar having an enclosed collar wadding material and forming a quilted surface engagement side, and a strap having an enclosed strap wadding material inner layer and a strap cover material. The strap is coupled to a back side of the quilted collar and extends from opposite ends of the quilted collar. One or more connectors are provided that taper at both distal ends, with the connectors coupled to the respective tapered distal ends of the strap. Each of the tapered connectors have a connector outer surface material in operative cooperation with the strap cover material, with the tapered connectors configured to pass through respective tracheostomy tube connectors and loop back to engage the strap material cover to securely fasten and position a tracheostomy tube when a tracheostomy tube is present.

In accordance with yet another embodiment, a method for assembling a tracheostomy tie is provided. The method comprises centering the major axis of a padded collar having a first major axis width with the major axis of a tapered strap that has a second width that is less than the first width. A padded collar is then coupled to the tapered strap, and one or more hook material connectors that taper on each distal end are coupled to respective distal ends of said padded collar.

These and other further features and advantages of the invention will be apparent to those skilled in the art from the following detailed description, taken together with the accompanying drawings, in which:

### BRIEF DESCRIPTION OF THE DRAWINGS

FIG. 1 is a perspective view of one embodiment of a tracheostomy tie according to the present invention;

FIG. 2 is another perspective view of the tracheostomy tie shown in FIG. 1;

FIG. 3 is another perspective view of the tracheostomy tie shown in FIG. 1;

FIG. 4 is another perspective and dimensional view of the tracheostomy tie shown in FIG. 1;

FIGS. 5a-c are cross section views of various embodiments through line 5-5 as shown in FIGS. 3 and 4;

FIGS. 6a-c are enlarged cross section views of various cover embodiments through lines 6a-6a, 6b-6b, 6c-6c shown in respective FIGS. 5a-c;

FIG. 7 is a plan view of another embodiment of a tracheostomy tie according to the present invention;

FIG. 8 is another perspective view of the tracheostomy tie shown in FIG. 1;

FIG. 9 is a dimensional view of one embodiment of a tracheostomy tie strap according to the present invention;

FIGS. 10a-c are perspective views of various embodiments of tracheostomy tie straps according to the present invention; and

FIG. 11 is a flow diagram describing assembly of one embodiment of a tracheostomy tie according to the present invention.

### DETAILED DESCRIPTION OF THE INVENTION

The following description presents several possible embodiments. This description is not to be taken in a limiting sense, but is made merely for the purpose of describing the general principles of the invention, the scope of which is further understood by the appended claims.

The components in the figures are not necessarily to scale, with emphasis instead being placed on illustrating the principals of the invention. Like reference numerals designate corresponding parts throughout the different views.

The present invention generally provides a tracheostomy tie and method which allows for a tracheostomy tube assembly to be efficiently, comfortably, and securely attached to a patient. Advantages of the tracheostomy tie and method include, but are not limited by, the following: durability, ease of attachment and removal, secure attachment, adjustability, being inexpensive to manufacture, and moisture management.

In accordance with one embodiment of the invention, a padded collar is provided that is preferably sewn to a strap on a side opposite from the patient neck engagement side, with the strap extending from opposite ends of the padded collar. The strap is preferably made of a loop material outer surface such as double-sided brushed polyester fiber material to work cooperatively with VELCRO™ hook material, and is tapered on its ends. A connector, preferably formed of VELCRO™ hook material, may be sewn to each end of the strap so the tapered connectors are operable to pass through respective tracheostomy tube assembly connectors (typically eyelets) and back to engage the loop material (See FIG. 2) so the padded collar securely fastens and positions a tracheostomy tube against the patient's neck. The connector is preferably tapered on both ends so it and the strap pass more easily through tracheostomy tube assembly connectors.

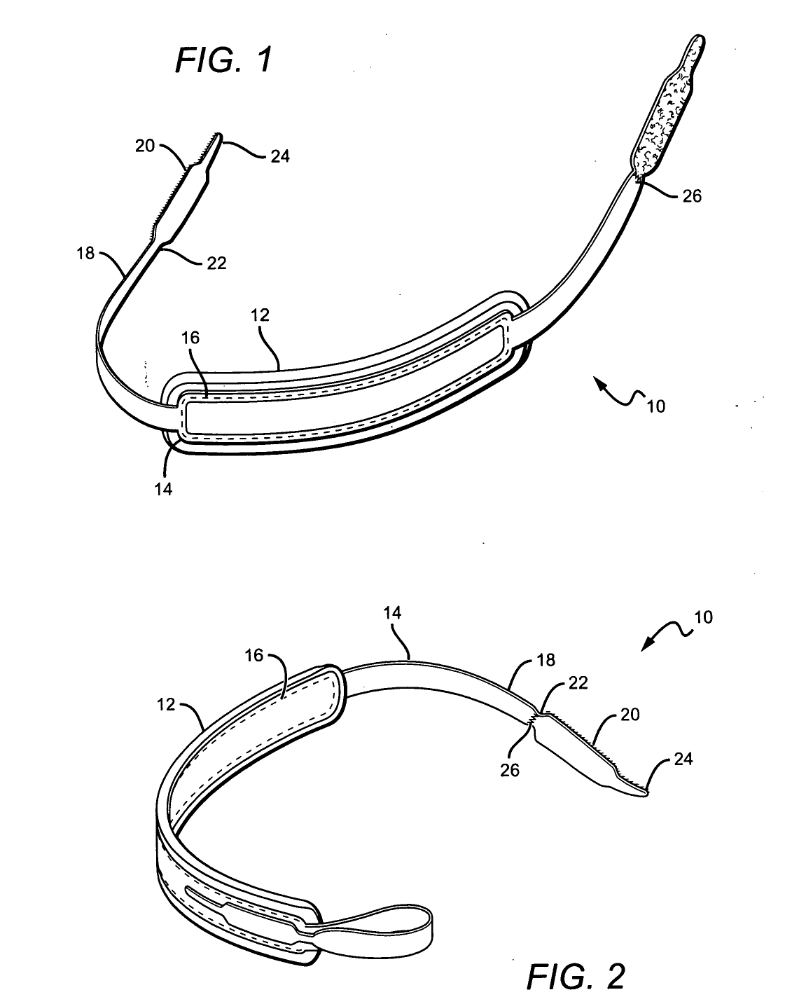

In one embodiment of the invention shown in FIGS. 1-2, a tracheostomy tie 10 has a padded collar 12 coupled to a strap 14, preferably sewn together adjacent longitudinal and lateral sides of the padded collar 12 as indicated with a dashed sew line 16. Although the padded collar 12 and strap 14 are illustrated sized and sewn together at a position set back from a perimeter of the padded collar, the strap 14 may be sewn with other patterns or immediately against the outer perimeter of the padded collar 12. The padded collar 12 and strap 14 may also be coupled using other suitable means, such as with fabric adhesive, by using fusible web techniques, or through the use of fusible material or other flexible glues and tapes.

The strap 14 extends beyond both ends of the padded collar 12 for engagement with tracheostomy tube assembly connectors, with the assembly connectors typically configured as eyelets (not shown). The strap 14 has at least a portion of its outer surface preferably formed of a looped material for complementary operation with a hook material connector. The strap 14 preferably has tapered portions on both of its respective distal ends 18 to aid in insertion through tracheostomy tube assembly connectors, with each distal end 18 also coupled (preferably sewn) at coupling points 26 to tapered connectors 20, the connectors 20 preferably made of hook material.

If loop material is used for the outer surface of strap 14, then hook material would be used for the tapered connectors 20. Similarly, if hook material is used for the outer surface of strap 14, then loop material would be used for the tapered connectors 20. The hook and loop material combination is commonly known as VELCRO™, with each material capable of coupling to the other. The tapered connectors 20 are preferably sewn to the distal ends 18 at coupling points 26 using overlap stitching to reduce any opportunity for the tapered connectors 20 to snag as the tapered connectors 20 of the tracheostomy tie 10 are attached to or removed from the tracheostomy tube connectors (not shown) during use. Alternatively, the tapered connectors 20 may be sewn or otherwise attached directly onto and ride the distal ends 18 of strap 14 rather than extend away from them.

The connectors 20 preferably comprise first tapered end 22 and second tapered end 24, with first tapered end 22 connecting to the tapered distal ends 18 of the strap 14 at coupling point 26. The portion of connector 20 in between first and second tapered ends 22 and 24 is preferably substantially wider than ends 22 and 24, with the widest portion of connector 20 advantageously providing a surface area large enough to ensure adequate connection with strap 14. Second end 24 is preferably narrow enough to be efficiently thread through a corresponding connector in a tracheostomy tube (not shown), with the motion of pulling second end 24 through the tube connector(s) allowing the wider portion of connector 20 to fold such that the hook material faces the inside of the fold. In this way, any resistance from the hook material as the connector is thread through a tube connector is minimized. First end 22 is preferably narrow enough to allow efficient removal of the tie 10 as the strap 14 and connector 20 pass back out of the connector(s) of a tracheostomy tube.

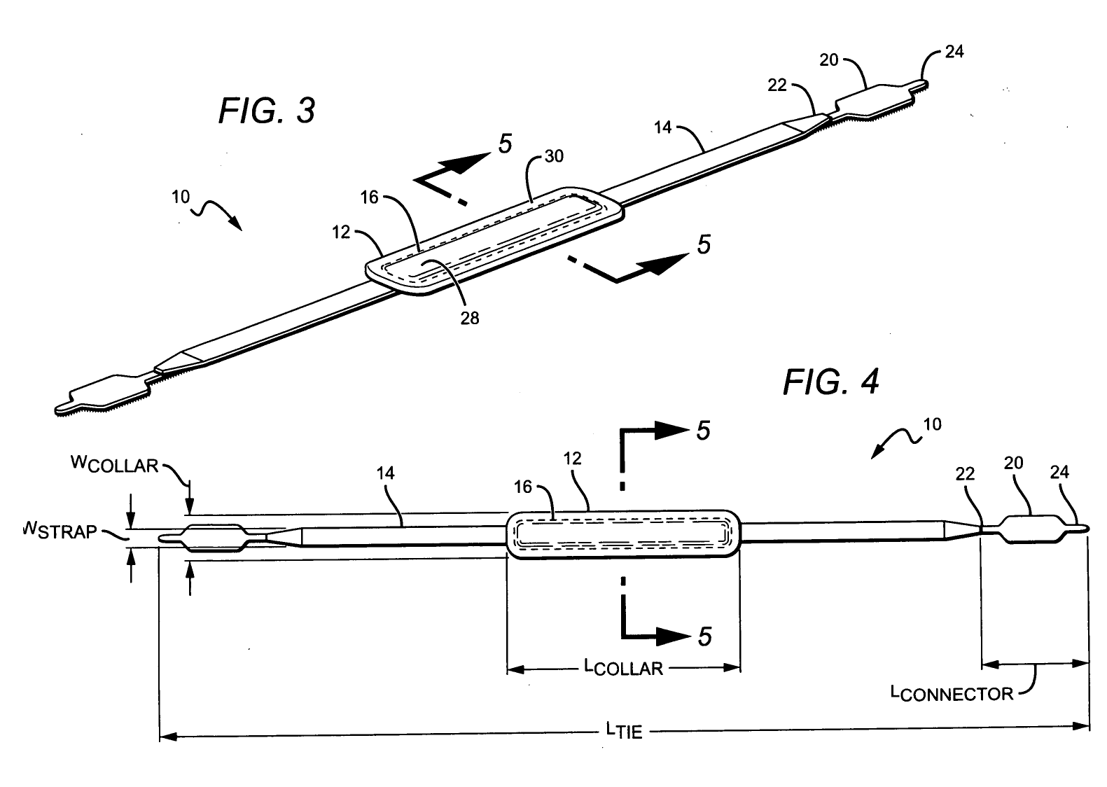

FIGS. 3-4 are perspective views of a tracheostomy tie 10 according to one embodiment of the present invention, illustrating a surface engagement side. The surface is preferably the neck of a patient requiring a tracheostomy tube. The strap 14 extends from opposite ends of the padded collar 12. The padded collar 12 is preferably sewn to the strap 14 at a location illustrated with dashed sew line 16 to establish a quilted exterior surface for comfortable engagement of a patient's neck. Although such a coupling establishes one longitudinal center pillow 28 and circumferential pillow 30, other quilted patterns may be established with a different sewing pattern. For example, dashed sew line 16 may form other patterns to establish a plurality of pillowed sections. Also, although the padded collar 12 is illustrated as generally rectangular with curved distal ends, the padded collar 12 may have square distal ends or may be itself polygonal or arcuate. In one tracheostomy tie having a θ_{TIE} of approximately 5 degrees and designed for use by a majority of patients, the various elements have the approximate widths and lengths listed in Table 1.

TABLE 1

| | Length (cm) |
|---|---|
| L_{TIE} | 29-62 |
| L_{COLLAR} | 15-30 |
| L_{CONNECTOR} | 7 |
| W_{COLLAR} | 2 |
| W_{STRAP} | 1 |

In an alternate embodiment, the padded collar 12 may be defined by more than one separate collar (not illustrated) to securely and comfortably attach a tracheal tube to a patient's neck.

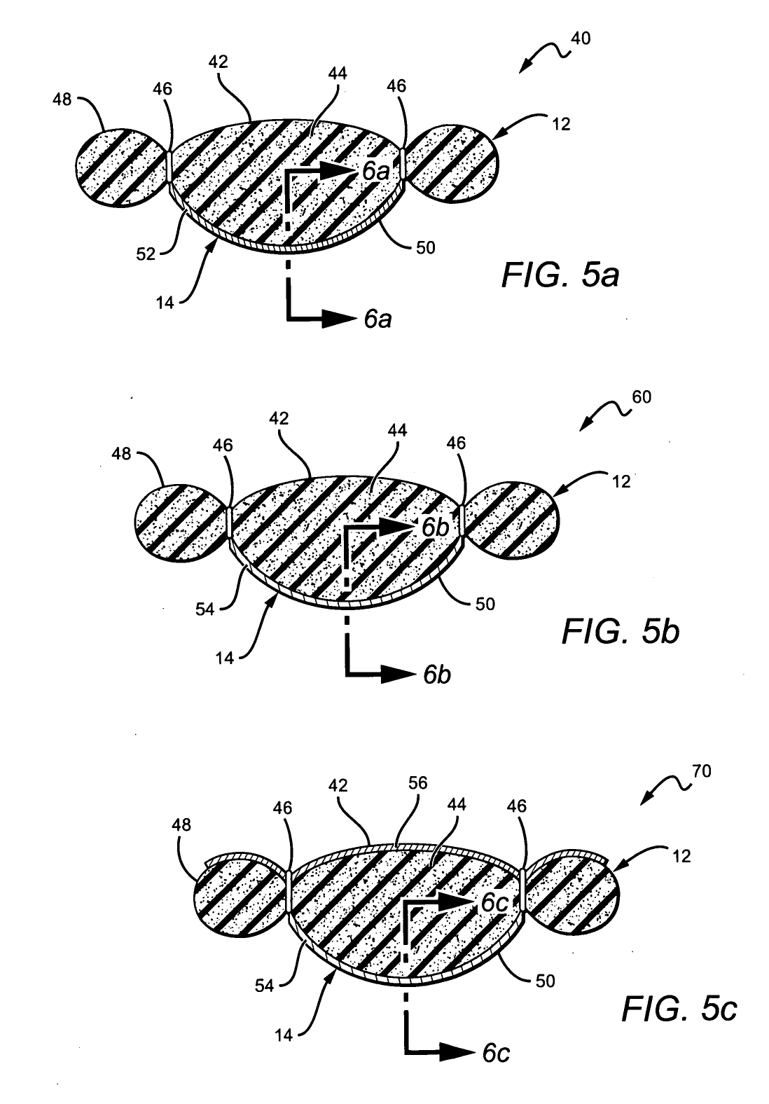

FIGS. 5a-c illustrate cross sectional views for alternative embodiments of the tracheostomy tie of FIGS. 3-4 as seen along the line 5-5. In FIG. 5a, a collar cover 42, preferably a soft cotton material, encompasses a collar wadding material 44, preferably padded foam, to establish the padded collar 40. In the embodiment illustrated in FIG. 5a, stitching 46 draws opposite sides of the padded collar 40 together to form a quilted exterior surface 48 that provides a comfortable engagement of a patient's neck without a patient's neck riding on a seam. Also coupled to the padded collar 40 by way of stitching 46 is the strap 14 defined by a strap wadding material, preferably foam material inner layer 52, enclosed by a laminated cover material 50, such as double-sided brushed polyester fiber. As the strap 14 experiences stress and resultant strain deformation during use, the collar wadding material 44 enclosed in collar cover 42 serves to resist induced bending moments to reduce changes in the shape of the quilted exterior surface 48.

In other embodiments, such as those shown in FIGS. 5b-c, the padded collar 40 has different materials as between the quilted exterior surface 48 and the side opposite the strap 14. For example, in the padded collar 60 of FIG. 5b, a collar cover 42 encompasses a collar wadding material 44 as in collar 40. However, strap 14 is defined by a different inner layer of material, preferably a moisture wicking material 54 such as rayon, enclosed by a laminated cover 50. In this way, strap 14 comprises wicking properties that aid in moisture maintenance that is commonly needed in patients requiring tracheostomy tubes and ties.

Alternatively, in the padded collar 70 of FIG. 5c, a collar cover 42 encompasses a collar wadding material 44 and a moisture wicking material 56 such as rayon. Strap 14 is defined by a wicking material 54 such as rayon, which is enclosed by a laminated cover 50. In this way, both the strap and the portion of the collar against the neck of a patient comprise wicking properties to aid in moisture maintenance and, ultimately, the comfort of the individual using the tracheostomy tie. Although these embodiments represent several possible compositions of the padded collar portion of the tie, it is understood that various combinations thereof or alternate configurations may also be contemplated within the scope of the present invention.

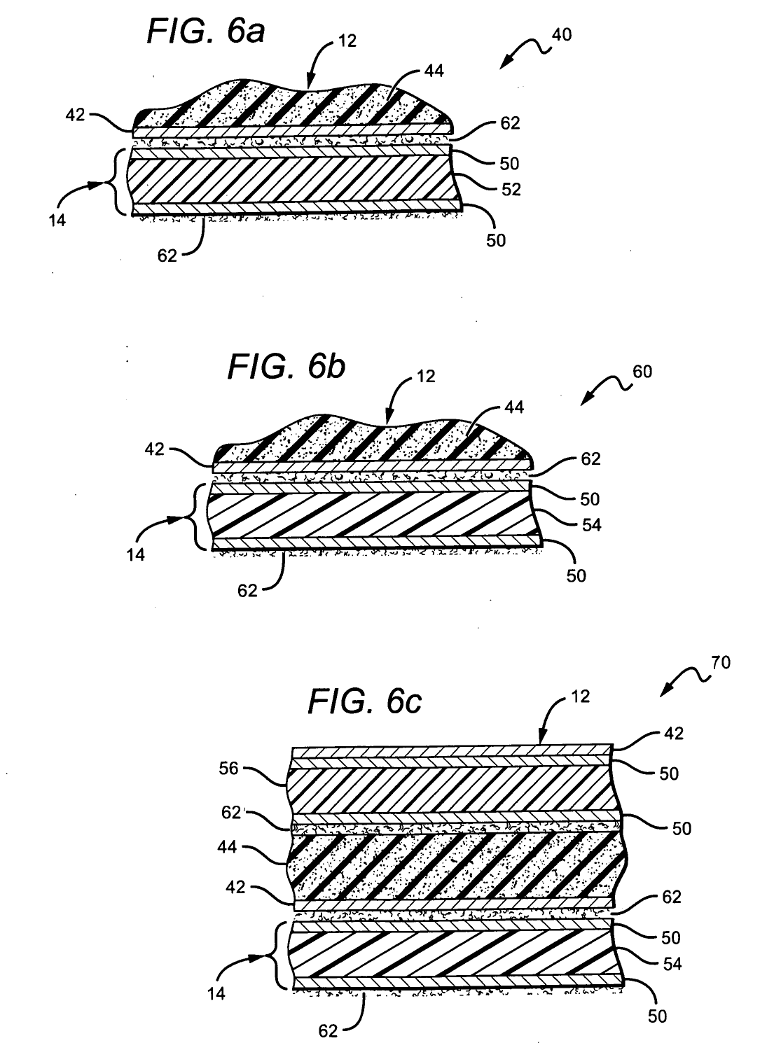

FIGS. 6a-c are enlarged cross sectional views of FIGS. 5a-c, respectively, as seen along respective lines 6a-6a, 6b-6b, and 6c-6c. In collar 40 of FIG. 6a, the foam material inner layer 52 of the strap 14 is enclosed by laminated cover 50. The laminated cover 50 of the strap 14 is illustrated with loops 62 on its exterior surfaces to illustrate possible complementary operation with hook material on the tapered connector 20 (not shown). Collar wadding material 44 is illustrated adjacent the collar cover 42.

In FIG. 6b, an enlarged cross sectional view of collar 60 is depicted. The wicking inner layer 54 of the strap 14 is enclosed by laminated cover 50. The laminated cover 50 of the strap 14 is illustrated with loops 62 on its exterior surfaces to illustrate possible complementary operation with hook material on the tapered connector 20 (not shown). Collar wadding material 44 is illustrated adjacent the collar cover 42. In FIG. 6c, collar 70 is depicted with a strap 14 similar to the strap of collar 60. Additionally, collar 70 has a collar cover 42, covering collar wadding material 44 and wicking layer 56. Wicking layer 56 is enclosed by laminated cover 50.

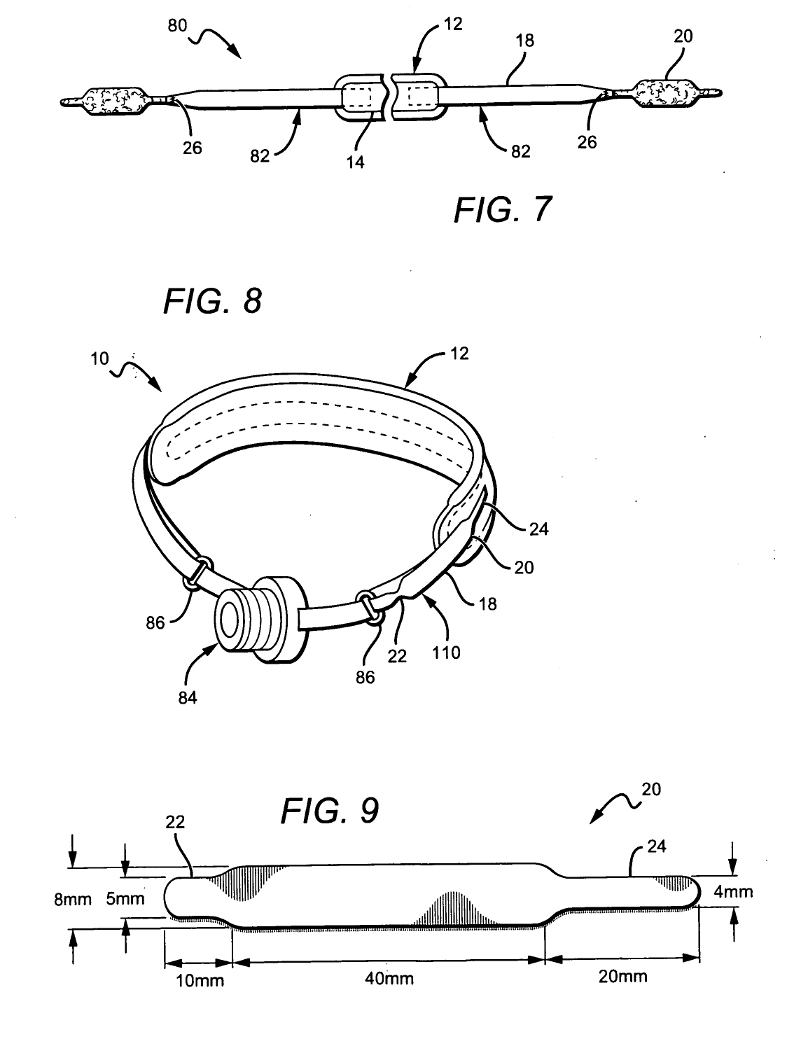

In FIG. 7, an alternative embodiment of a tracheostomy tie according to the present invention is illustrated. In tie 80, the strap 14 is defined by two non-contiguous strap assemblies 82 coupled, preferably sewn, to the padded collar 12 to reduce the quantity of utilized material. In this embodiment, tapered connectors 20 are also preferably attached to the strap assemblies 82 with the overlap stitch and coupling portion 26 illustrated in FIG. 1 to reduce any opportunity for the tapered connectors 20 to snag as the tapered connectors 20 are removed from the tracheostomy tube connectors (not shown) during use.

FIG. 8 illustrates a tracheostomy tube assembly assembled with the tracheostomy tie 10 depicted in FIGS. 1 and 2. Tapered connectors 20 and distal ends 18 of the strap 14 have been inserted into tracheostomy tube connectors 86 (illustrated as eyelets) and looped back to engage with the loop surface of the padded collar 12. Tapered portions 22 and 24 of the tapered connector 20 and distal ends 18 facilitate easy insertion of the strap 14 through the tube connectors 86, while overlap stitching and coupling portions 26 facilitate efficient removal of the same. The strap 14 is preferably sewn to the padded collar 12 at points set back from an outer perimeter of the padded collar 12 to draw the padded collar 12 against a user's neck as the tracheostomy tie is positioned and fastened.

FIG. 9 depicts a dimensional view of one alternative embodiment of a tapered connector 20 according to the present invention. As indicated, in a preferred embodiment of a connector 20 according the present invention, connector 20 is substantially wider in its middle portion than it is at either of first and second tapered ends 22 or 24. As shown, connector 20 may be ˜8.0 mm wide at its widest point, with first and second ends 22 and 24 being ˜5.0 mm and 4.0 mm wide respectively. The widest part of connector 20 is ˜40.0 mm in length, with ends 22 and 24 being ˜10.0 mm and ˜20.0 mm respectively. The total length of connector 20 is thus ˜70.0 mm. While FIG. 9 depicts one possible dimensional arrangement of a connector 20, it is understood that other dimensions are contemplated in the scope of the present invention.

Second tapered end 24 is preferably wide enough so it readily fits through tracheostomy tube connectors (best shown in FIG. 8 as eyelets 86). As connector 20 widens from second end 24 to the widest point of its middle portion, it preferably does so with a radius of curvature (in this embodiment, ˜4.0 mm) that forces the middle portion to fold in on itself such that the hook side of the material is toward the inside of the fold. This allows the connector 20 to move more easily through a tube connector by minimizing the resistance of the connector 20 material against the tube connector. Once the widest portion of connector 20 is passed through the tube connector, it is looped back to connect with the loop material of the strap 14. The larger width and length of the middle portion provides greater surface area of hook material to connect with the loop material, enabling a secure means of fastening a tracheostomy tie with a tracheostomy tube. Additionally, the tapering of strap distal end 18 and connector 20 to first end 22 allows strap 14 and connector 20 to easily pass back out through a tube connector with minimal resistance.

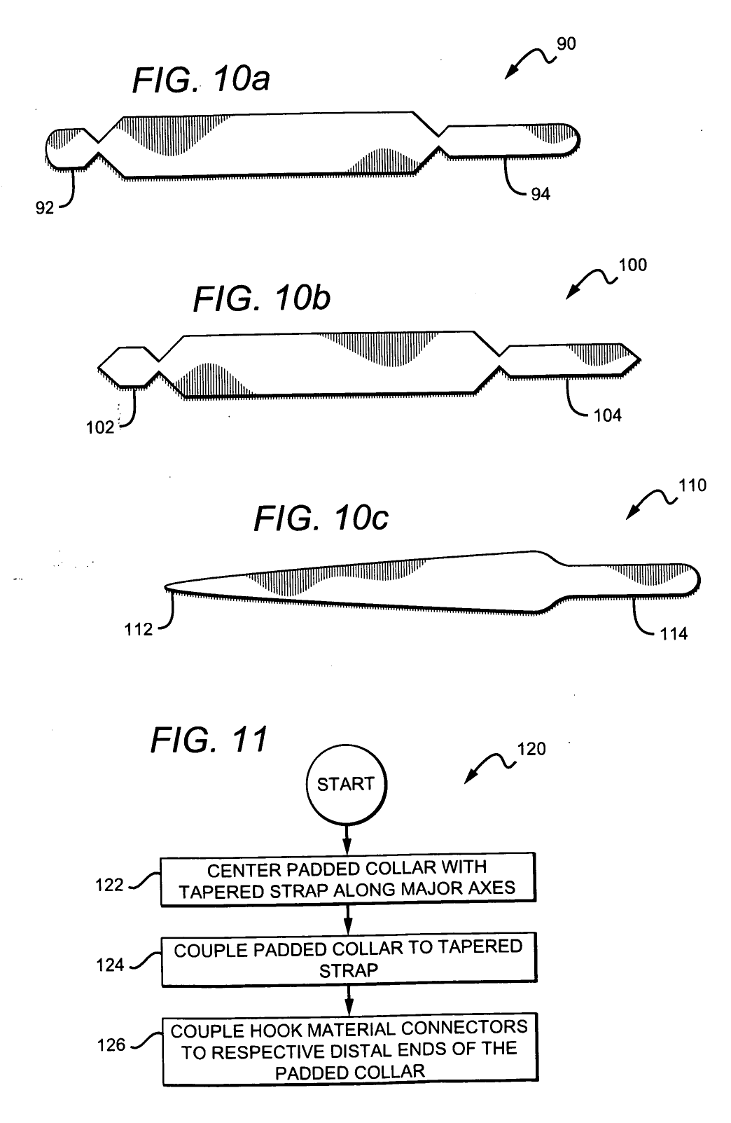

FIGS. 10a-10c illustrate several additional embodiments for a connector with tapering on both of its respective distal ends. FIG. 10a depicts connector 90, which has rounded first and second tapered ends 92 and 94. As shown, the transition from first and second tapered ends 92 and 94 to the widest portions of connector 90 comprises a pointed, diamond-like transition rather than the curved transition of connector 20. FIG. 10b depicts connector 100 with first and second pointed tapered ends 102 and 104 that have pointed transitions similar to that of connector 90. FIG. 10c depicts connector 110, which has an end 112 which tapers gradually to a point from the widest portion of connector 110. It also comprises a rounded end 114, which comprises a curved transition similar to that of connector 20. While connectors 20, 90, 100, and 110 depict several possible tapered connectors according to the present invention, it is understood that various additional combinations and configurations of connector portions are contemplated within the scope of the present invention.

FIG. 11 illustrates one embodiment of a method for assembling a tracheostomy tie. Major axes of the padded collar and tapered strap are centered (block 122) and the components are coupled together (block 124), preferably sewn together, to create a quilted section on the padded collar. The quilted section preferably forms center and peripheral pillow surface areas so a patient's neck rides on the pillows and is not in contact with the seam during normal use. The pillow surface also has the added benefit of allowing better air circulation between a patient's neck and the tracheostomy tie to reduce neck breakdown during normal use.

Tapered connectors are coupled to respective distal ends of the padded collar (block 126), preferably with a flush seam. The flush seam creates a smooth transition between the hook material connectors and the padded collar to reduce the possibility of snagging the hook material connectors on a tracheostomy tube connector when a caregiver is removing the hook material connector from the tracheostomy tube connector. The unique combination of a quilted padded collar, tapered strap, tapered connectors and smooth seam stitching between the strap and connectors address both the real-world problem of patient skin breakdown underneath tracheostomy ties during their extended wear and the physical challenge faced by a single caregiver as he or she changes out a tracheostomy tie by him or herself.

Although the present invention has been described in considerable detail with reference to certain preferred configurations and methods, other versions are possible. The invention can be used in many different scenarios where a tie is needed to fasten a connectable device to an intervening person or object. Different materials, material configurations, tapered connectors, stitching styles, and straps can be used, and a variable number of connectors can be used to provide for any number of corresponding connection points on a connectable device. Additionally, any combination of tapered connectors, straps, materials and the like may be used to achieve a tracheostomy tie according to the present invention. Therefore, the spirit and scope of the above description should not be limited to the versions described above.

I claim:

1. A tracheostomy tie apparatus, comprising:
a padded collar comprising a surface engagement side; and
a strap coupled to said padded collar on a side opposite from said surface engagement side and extending from opposite ends of said padded collar, said strap comprising:
a loop material outer surface and tapered distal ends; and
one or more connectors tapered at both distal ends, said connectors coupled to respective tapered distal ends of said strap, each of said tapered connectors having a hook material outer surface;
wherein said one or more connectors are operable to pass through respective tracheostomy tube connectors and loop back to engage said loop material so said padded collar securely fastens and positions a tracheostomy tube.

2. The apparatus of claim 1, wherein said padded collar comprises:
a collar wadding material; and
a collar cover;
wherein the combination of said collar wadding material and said collar cover provide soft engagement on said surface while resisting bending moments created in said strap in response to stress-strain experienced by said strap during use.

3. The apparatus of claim 2, wherein said collar wadding material comprises a foam material.

4. The apparatus of claim 2, wherein said collar cover comprises a soft cotton material to provide a wicking capability for said surface.

5. The apparatus of claim 2, wherein said collar further comprises a layer of wicking material outside of said wadding material and adjacent to said surface.

6. The apparatus of claim 5, wherein said wicking material comprises rayon.

7. The apparatus of claim 2, wherein said collar cover sewn together adjacent longitudinal sides to enclose said collar wadding material.

8. The apparatus of claim 1, wherein said surface engagement side comprises:
a quilted exterior surface to provide comfortable engagement of the patient's neck.

9. The apparatus of claim 1, wherein said strap is coupled to said padded collar on said surface engagement side at a plurality of coupling points set back from an outer perimeter of said padded collar.

10. The apparatus of claim 1, wherein said one or more tapered connectors are coupled to said respective tapered distal ends with an overlap stitch so said connectors do not snag said tracheostomy tube connector when attached and/or removed.

11. The apparatus of claim 1, wherein said one or more tapered connectors are widest in the middle such that substantial surface area is provided for secure attachment to said loop material.

12. The apparatus of claim 1, wherein said one or more tapered connectors are narrow enough on said tapered ends so a said connector can readily pass through a respective tube connector.

13. The apparatus of claim 12, wherein the transition from said tapered ends to the middle of a said connector enables a said connector to fold such that said hook material is on the inside of said fold and the entire connector can readily pass through a respective tube connector.

14. The apparatus of claim 1, wherein said strap further comprises a laminated cover to provide a soft engagement with said surface.

15. The apparatus of claim 14, wherein said laminated cover comprises a double-sided brushed polyester fiber material to wick moisture from said surface.

16. The apparatus of claim 15, wherein said strap further comprises a wadding material inside said laminated cover, said wadding material comprising a foam, rayon, or other wicking material.

17. A tracheostomy tie apparatus, comprising:
a padded collar having an enclosed collar wadding material and forming a quilted surface engagement side;
a strap having an enclosed strap wadding material inner layer and a strap cover material, said strap coupled to a back side of said quilted collar and extending from opposite ends of said quilted collar; and
one or more connectors tapered at both distal ends, said connectors coupled to respective tapered distal ends of said strap, each of said tapered connectors having a connector outer surface material in operative cooperation with said strap cover material, said tapered connectors configured to pass through respective tracheostomy tube connectors and loop back to engage said strap cover material to securely fasten and position a tracheostomy tube when a tracheostomy tube is present.

18. The apparatus of claim 17, wherein said strap further comprises two tapered distal ends.

19. The apparatus of claim 17, wherein said strap is coupled to said quilted collar on said surface engagement side at a plurality of coupling points set back from an outer perimeter of said padded collar.

20. The apparatus of claim 17, wherein said strap is formed from at least two coupled sections, each of said at least two sections coupled to opposite ends of said padded collar.

21. The apparatus of claim 17, wherein said enclosed collar wadding material is padded foam, rayon and/or another wicking material.

22. The apparatus of claim 17, wherein said enclosed strap wadding material is padded foam, rayon and/or another wicking material.

23. A method of assembling a tracheostomy tie, comprising:
centering the major axis of a padded collar having a first major axis width with the major axis of a tapered strap that has a second width that is less than said first width;
coupling said padded collar to said tapered strap; and
coupling one or more hook material connectors that taper on each distal end to respective distal ends of said padded collar.

24. The method of claim 23, wherein coupling said padded collar to said tapered strap comprises sewing said padded collar to said tapered strap to create a quilted section.

25. The method of claim 23, wherein said coupling hook material connectors further comprises sewing said hook material connectors to respective strap distal ends with a flush seam to create a smooth transition between said hook material connectors and said padded collar to reduce the possibility of snagging said hook material connectors on a tracheostomy tube connector when removing said hook material connector from said tracheostomy tube connector.
